(19) United States
(12) Patent Application Publication
(10) Pub. No.: US 2020/0229701 A1
(43) Pub. Date: Jul. 23, 2020

(54) Tracing Device and a Tracing Method

(71) Applicant: NANJING NUOYUAN MEDICAL DEVICES CO., LTD., Nanjing (CN)

(72) Inventors: Huiming Cai, Jiangsu (CN); Yiqing Wang, Jiangsu (CN)

(21) Appl. No.: 16/417,318

(22) Filed: May 20, 2019

(30) Foreign Application Priority Data
Jan. 18, 2019 (CN) CN201910051685.5

(51) Int. Cl. A61B 5/00; A61B 90/00

(52) U.S. Cl. CPC A61B 5/0071; A61B 5/0077; A61B 90/39

(57) ABSTRACT

Provided are a tracing device and a tracing method which relate to the field of medical equipment. The tracing device is used to manifest the marker in the detected subject. It includes a light source and an optical identifier. In the above, the light source is configured to provide a first beam to irradiate the detected subject. And the first beam can interact with the detected subject and then the detected subject generates a second beam different from the first beam. In the above, the optical identifier is configured to detect the second beam so as to identify the marker on the detected subject. The tracing device of the present disclosure is highly sensitive and has simple structure. The tracing method of the present disclosure is easy to implement and can clearly identify markers.

### CROSS REFERENCE TO RELATED APPLICATIONS

The present disclosure claims the priority to the Chinese Patent Application (No. 2019100516855), entitled “A Tracing Device and a Tracing Method”, filed with CNIPA on Jan. 18, 2019, the entirety of which is incorporated herein by reference.

### TECHNICAL FIELD

The present disclosure relates to the field of medical equipment and specifically to a tracing device and a tracing method.

### BACKGROUND ART

Currently, for example in angiography, we often need the help of an optical system to observe the blood vessels in the subject. However, the images of pathological tissues obtained by the existing optical auxiliary equipment are not clear enough and go against consecutive surgical operation.

The information disclosed in the part of the background art is only intended to enhance the understanding of the overall background art of the present disclosure, but should not be construed as acknowledging or implying in any way that such information constitutes the prior art well known to those skilled in the art.

### SUMMARY

Embodiments of the present disclosure provides a tracing device, configured to showing markers in the detected subject.

The tracing device includes a light source and an optical identifier.

In the above, the light source is configured to provide a first beam to irradiate the detected subject, and the first beam can interact with the detected subject and the detected subject generates a second beam different from the first beam.

In the above, the optical identifier is configured to detect the second beam so as to identify the markers on the detected subject.

Embodiments of the present disclosure further provides a tracing method which can be implemented by the tracing device as described above, wherein the tracing method includes:

using the first beam emitted from the light source to irradiate the detected subject, in which case a tracer in the detected subject can generate a second beam under the excitation of the first beam; and

receiving, by the optical identifier, the second beam generated by the tracer in the detected subject so as to show an image of the marker different from the detected subject.

### BRIEF DESCRIPTION OF DRAWINGS

In order to more clearly illustrate the technical solutions in the embodiments of the present disclosure or in the prior art, drawings required to be used in the description of the embodiments or the prior art will be briefly introduced below.

FIG. 1 is a simplified structural schematic diagram of a tracing device provided by an embodiment of the present disclosure;

FIG. 2 illustrates a schematic flow diagram of a tracing method provided by an embodiment of the present disclosure;

Reference signs: 100-tracing device; 1-optical identifier; 2-light filter; 3-anti-reflection lens; 4-light provider; 5-filter; 6-marker; 7-detected subject.

### DETAILED DESCRIPTION OF EMBODIMENTS

The implemented scheme of the present disclosure will be described below in detail with reference to embodiments. But those skilled in the art will understand that the following embodiments are used only to illustrate the technical content of the present disclosure, and should not be construed as limiting the scope of the present disclosure. Embodiments for which no specific condition is indicated should be done under conventional conditions or conditions as recommended by the manufacturer. The reagents or instruments used for which no manufacturer is indicated are all conventional products which are commercially available.

The tracing device and the tracing method of the embodiments of the present disclosure will be detailed below.

In medical activities, in order to obtain the image of lesions or know their situation, such as distribution, tissue morphology and the like, usually we need to perform the invasive methods (e.g. surgery) to expose them and view them directly with naked eyes. Or, we use modernized medical equipment e.g. CT (Computed Tomography), X-ray and MRI (Magnetic Resonance Imaging) etc. to take pictures in a non-invasive way and take records of relevant image information.

Some of the above measures are widely used, but they have obvious defects.

For example, invasive surgical operations often leave permanent or lasting wounds on patients which may aggravate or worsen the patients' condition. Some postoperative complications may also interfere with subsequent medical measures.

Or, in some other measures from the above, although, generally, for example surgical treatment is not required for the patients, they normally will take a certain degree of (or even a high dose of) radiation which may cause potential safety risk or hidden danger. This is especially prominent when patients suffer from severe diseases. In addition, such non-invasive measures also usually require quite precise and dedicated equipment which in most cases is expensive and hard to operate and maintain.

Further, in order to obtain image information, such equipment normally needs to be provided with dedicated image processing equipment and to be combined with the assistance of computer software to complete information processing. This makes it even more difficult to obtain images from this and is not user-friendly for those medical staff less competent in medical treatment. In other words, in order to obtain corresponding information from those images accurately, rich experience and profound professional skills are required from medical care staff.

In view of the above, the inventor proposes a tracing device 100 and a tracing method, which are used to identify the target object from the subjects of interest.

For example, a particular object is identified directly from human body or in vitro human tissues, cells (e.g. tumor cells and cancer cells), organs and the like. Of course, other than some of the above subjects (i.e. human), non-human animals are also possible, including but not limited to mammals, such as dog, cat, horse, monkey, rabbit, cow, pig, sheep, goat, rat, mouse, guinea pig, hamster, fish, bird, amphibians, primates and the like. The target object may be for example a tumor, a pathological tissue or the like.

For those skilled in the art to understand better, as an instance, detection of human tumor will be illustrated below.

When the detection is performed, the use of tracer is involved. In the above, the tracer is a substance described below.

First, the tracer is not significantly toxic or irritative and can well adapt itself to in vivo or in vitro tissues, cells and organs, etc., or has a good/desired yield/risk ratio. That is, ideally, the tracer does not induce medically undesired excessive irritant or toxic effect in the subject, or it may cause certain irritation or poisoning to the subject, but remains within a controllable range or an acceptable degree.

Second, the tracer normally is also expected to be able to stay in the body of the subject for a suitable period of time, so as to perform and complete the detection operation. The tracer may suffer from some metabolic loss in the in vivo or in vitro subject. Therefore, it is not expected to vanish completely within a short time as a result of metabolism. On one hand, the tracer per se, desirably, should not be transformed or excreted by the subject. On the other hand, if it is transformed or excreted to a certain degree, it is important to control its dose. For example, in its desired retention time, we may increase the initial dose so that the retention amount after consumption within the subject may meet the testing requirement.

Third, in the tracing device 100 and method in the present disclosure, the tracer is also expected to be able to luminesce at excitation. That is, the tracer can be excited by light and thus generate light rays, i.e., photoluminescence. Further, as the light rays generated by excitation are different from the exciting light, the exciting light and the excited light can be distinguished in a proper way.

In the examples of the present disclosure, optionally, indocyanine green (ICG, CAS No.: 3599-32-4) is chosen as the tracer and the detected subject 7 is human. The target object is a tumor tissue. Indocyanine green can be phagocytized by the tumor tissue but its metabolic rate is relatively slow. Therefore, indocyanine green (ICG) will not be metabolized completely out of the tissue by the human body within a certain period of time. Thus, a certain amount of indocyanine green (ICG) will remain in the tumor tissue within a certain period of time. Indocyanine green, as a tracer, may also be called as contrast medium and used in this way.

The retention time of the indocyanine green in the human body may be obtained by measuring the retention rate in the blood or the blood plasma disappearance rate by intravenous injection. For example, an indocyanine green drug is diluted with sterilized water for medical injection and then injected from the cubital vein. Then we should measure its content in the cardiac output. Or, we measure the hepatic blood flow by intravenous drip.

In practice, indocyanine green is prepared in solution at a certain concentration and then injected into the subject. In other embodiments of the present disclosure, other than introducing indocyanine green into the subject by way of injection, indocyanine green may also be introduced into the subject by other medically acceptable ways.

In order to avoid unrecoverable damage to the human body (especially of the severe diseases), in the example, in vitro tissues from the human body may be used for experiments.

Alternatively, indocyanine green is prepared in mixed solution containing a certain concentration of indocyanine green (ICG). The solution has graded concentrations, respectively, for example 10-6, 10-7, 10-8, 10-9 and 10-10 etc. In the tissue to be tested, indocyanine green is introduced into the tissue by injecting a certain dose of indocyanine green (ICG). After a certain period of time, the tissue to be tested is irradiated by a light source. Due to properties of tracer indocyanine green, it will generate fluorescence after irradiated by light with certain wavelength. Thereby, information about the tumor may be obtained by detecting the fluorescence. For example, if the detected subject 7 has a tumor, the fluorescence may show information about the tumor e.g. its location, shape and volume. If the detected subject 7 has no tumor, the detected subject 7 does not show fluorescence, based on which, to some extent, the existence of tumor cells may be excluded.

Based on this, the tracing device 100 and the tracing method proposed in the examples may be applied in clinical medicine. For example, the examples provide a diagnosing method of tumor. The diagnosing method includes the following steps. First, the prepared indocyanine green solution is injected into the human body. Second, after the patient has been staying still for a certain period of time, the diseased location (e.g. neck) of the patient is irradiated by a light source. Third, a fluorescence detector is used to take pictures of the diseased location to obtain images of the diseased location. From the images, doctors may know the condition of the tumor in the patient, and in conjunction with, for example, blood detection, histological anatomy and case analysis, the doctor will be able to develop a corresponding therapeutic regimen for the patient. In making diagnosis, normally the detection results are required to be analyzed and compared (e.g. compare with normal data).

The above content is given as unlimited cases of the examples, which acts as a recapitulative description. The tracing device 100 and method will be explained below and relevant and more detailed description will also be involved.

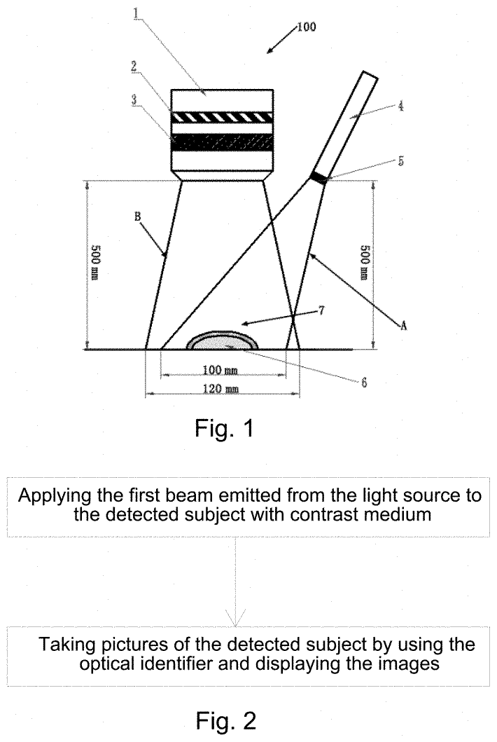

Referring to FIG. 1, in general, the tracing device 100 in the embodiments of the present disclosure may be used to manifest the marker 6 in the detected subject 7. In the above, the detected subject 7 may be human or non-human animal, or also may be in vitro tissues, cells, organs and the like of the human or non-human animal. The marker 6 may be for example a tumor, a pathological tissue or the like. In the above, the marker 6 is normally compatible with the tracer as described above. In other words, based on the device and method in the embodiments of the present disclosure, when the tracer is determined, the detected subject 7 and marker 6 it can handle normally are a definitive category which may include one or more types. Or, when the detected subject 7 and marker 6 are determined, the tracer may be the set of some optional substances, or a composition having definitive components or a compound having a definitive structure, etc.

Exemplarily, the tracing device 100 includes a light source (which includes for example a light provider 4 and a filter 5) and an optical identifier 1. Normally, based on the consideration of convenience for users, the light source and the optical identifier 1 are expected to be provided in the form of an integral or integrated device or equipment. Therefore, in such examples, the tracing device 100 may need to be equipped with, for example, a frame (a holder, a base or a seat) or the like, so that the light source and the optical identifier may be assembled/mounted or fixed by the frame. Based on the implementing mode of the light source and the optical identifier, they may be directly or indirectly connected in various properly selected ways, e.g. welding, bolting, riveting and pivoting.

In the examples of the present disclosure, the light source and the optical identifier are provided and used as independent equipment. Both of them may be flexibly placed and used as required by the user.

In the above, the light source is configured to provide a first beam which irradiates the detected subject 7. The first beam may be selectively configured as required. For example, the first beam may be laser. Further, the first beam has a wavelength between 779 and 791 nm. More further, the first beam has a wavelength between 780 and 790 nm. It should be noted that the first beam does not have to be selected as laser, but may also be other forms of light. Its selection is mainly intended to be compatible with the tracer (to the extent that it can excite the tracer to luminesce). When the first beam is selected as laser, it may have various options for wavelength range. But considering that high energy impact to the detected subject 7 or laser production is hard, its wavelength value or range (which obviously should at least excite the tracer to luminesce) may be limited to 779˜791 nm, or 780˜784 nm, or 782˜785 nm, or 783˜786 nm etc.

Thereby, the first beam reaches the detected subject 7 in proper ways (perpendicular irradiating or obliquely irradiating the diseased location or the area to be detected) and can interact with the detected subject 7, in which case the detected subject 7 generates a second beam different from the first beam. Optionally, the second beam is fluorescence (which is of near infrared light). In some examples, the second beam has a wave peak with a central wavelength of 810 to 830 nm. In some other examples, the second beam has a wavelength of 810 to 820 nm. Therefore, the first beam and the second beam mainly differ in wavelength. For example, the first beam (exciting light) is laser, and the second light (excited light) is fluorescence.

In addition, it should be noted that the laser and fluorescence may be selected as visible light or invisible light as required. As the tracer may be excited to luminesce immediately after irradiation, generally regardless of the irradiation angle and direction, the first beam may irradiate the area to be detected of the detected subject 7 at various angles. And generally, the first beam may be selected to obliquely irradiate the area to be detected. Accordingly, the optical identifier may be selected to directly perpendicular to or directly facing the area to be detected. In such case where the optical identifier directly faces the area to be detected, relatively standard and high-quality images of the area to be detected may be obtained. This largely reduces cumbersome operations like image correction and registration which are required to be done for images at oblique angles obtained as the optical identifier obliquely points to the detected area. That is, relative to the area to be detected, the light source is oblique, and the first beam obliquely irradiates (is incident to) the detected subject 7, while the optical identifier is directly facing/perpendicular to the area to be detected, and the second beam is perpendicularly emitted (emergent) from the detected subject 7 and enters the optical identifier.

Various known suitable luminescent devices may be used to generate beams which are used as the light source, and then optional suitable light modulation is done for the purpose of providing the first beam. For example, when the luminescent device can directly generate the first beam as required, it may directly be used as the light source. If the luminescent device cannot meet the desired standard or requirement, then obviously, the light generated by the luminescent device needs control. For example, such light control may be wavelength filtering (to select light with a specific wavelength/frequency). Or, it could be the size of light. For example, the size of the light spot formed when the laser irradiates the detected subject 7 (certainly, which is expected to be able to cover the detected area). Or, it could be the energy of light, irradiation duration and frequency, etc.

From the above, as an example, the light source includes a light provider 4 and a filter 5 (e.g. light filter 2, band-pass light filter 2) compatible with each other. The filter 5 is configured to filter the light generated by the light provider 4 in terms of wavelength so as to generate the first beam. The filter 5 may be a single lens or the combination of a plurality of lenses or an independent device. Therefore, in some examples, the light source may include a (columnar) shell which accommodates in its interior the light provider 4 and the optional filter 5. The optional filter 5 is located within the shell and on the emergent light path of the light provider 4 so as to filter the light generated by the light provider 4. Further, the light source may further be provided with a power supply which is electrically connected with the light provider 4. For example, the power supply may be a power source (mains supply or battery-primary battery, secondary battery, lithium ion battery) or a power adapter.

Further, in practice, the area of the detected subject 7 that needs to be detected may be relatively large or relatively small (of course may also have e.g. an overall dimension with a size normally from 1 to 5 cm). In this case, the first beam emergent from the light source is expected to have an adjustable size (beam diameter, area of the light spot on the detected subject 7 when irradiated). As shown in FIG. 1, the light spot formed by the first beam A on the detected subject 7 has a size of 100 mm, and its emergent point may be 500 mm high from the detected subject. The inlet in the optical identifier for the second beam to enter may also be 500 mm high from the detected subject. In other words, the emergent height of the first beam (relative to the light source, as the light emitting hole of the emergent light head mentioned below) may be equal to the incident height of the second beam (relative to the optical identifier). In order to obtain more comprehensive information, the field of view B of the optical identifier may cover the light spot formed by the first beam A and may have an overlapping area therewith. The field of view B may have a size of 120 mm in the projection part on the surface of the detected subject.

As such, the tracing device 100 is expected to have an adjuster. In an example, the adjuster may make adjustment by changing the distance between the aforementioned light provider 4 and the detected area by moving the light provider (for example, by moving the shell that accommodates the light provider 4). That is, the adjuster may be a mechanical arm. The shell is fixed on the mechanical arm. The mechanical arm may move under the control of a control devices. Alternatively, the adjuster may be a lens which may be selected to (properly and controllably) condense (a convex lens) or diverge (a concave lens) the beam as required. The lens may be configured to be adjustable relative to the position of the light provider so that in use the distance between the lens and the light provider 4 may be adjusted in due time.

In addition, if the light source is expected to have a plurality of irradiation patterns, its emission patterns/modes are required to be adjustable. Therefore, the light source may also be provided with a controller. The controller may adjust its emission frequency by controlling the power on-off of the power supply unit. Alternatively, the controller may also change the size of the light spot etc. by controlling the movement of the aforementioned adjuster. As an industrialized control equipment, the controller may be various electronic parts and components capable of performing certain data storage and processing or the collection thereof. For example, it could be central processing unit (CPU), microcontroller unit (MCU), programmable logic controller (PLC), programmable automation controller (PAC), industrial control computer (IPC), field-programmable gate array (FPGA), application specific integrated circuit (ASIC chip), etc. Of course, as an upper computer, the controller also cooperates with the lower computer (an equipment for performing certain operations). The controller gives control instructions, and the lower computer executes the actions corresponding to such instructions.

For example, the controller may control the mechanical arm acting as the adjuster (an optional lower computer), in a way that the controller may control the rotation of the motor, the reduction ratio of the reducer or the telescoping of the hydraulic cylinder etc. in the mechanical arm. More specifically, the controller may control the gear of the reducer, the power output of the motor, etc.

Although the light source may have various optional structures and implementing modes as above, it should be appreciated that the light source may also be directly adjusted and configured beams/light rays. In addition, the first beam generated by the light source may also have various patterns of manifestation. The patterns of manifestation herein mainly refer to the shape of the first beam, e.g. point light, ceiling light, lattice light and linear light, etc.

As point light, the first beam forms a single light spot (e.g. having a circular, oval shape, etc.) on the detected subject 7. As ceiling light, the first beam forms a single light spot (which is normally larger than that in the case of point light, and has a circular, oval, rectangular, polygonal shape, etc.) on the detected subject 7. As lattice light, the first beam forms a plurality of light spots (at least two) on the detected subject 7. The plurality of light spots are distributed in a matrix, array or other patterns.

In order to obtain the first beam with the required pattern of manifestation, a corresponding emergent light head may be provided at the light source. For example, the emergent light head is a cylinder which is sleeved on the shell which accommodates the light provider 4. One end of the emergent light head has an opening (for screw-thread fit or clamping, etc.), and the other end has a light emitting hole. The way that the light emitting hole is arranged corresponds to the mentioned pattern of manifestation. For example, a single light emitting hole having a small diameter may correspond to point light. A single light emitting hole having a large diameter may correspond to ceiling light. A plurality of light emitting holes having a small or large diameter may correspond to lattice light.

In addition, it should be noted that based on respective safety requirements, industry standards and mandatory standards, etc., in some examples, the intensity of light source of the first beam generated by the light source may be required to be less than or equal to 0.499 W.

The above describes the light source and the components possibly selected to work with it. The optical identifier will be described below.

As aforementioned, the optical identifier is configured to detect the second beam so as to identify the marker 6 on the detected subject 7. In other words, when the detected subject 7 emits the second beam, the second beam may be identified by the optical identifier of the tracing device 100. And the second beam may manifest the marker 6 (e.g. the aforementioned tumor), for example the outline of the marker 6. The optical identifier may be configured properly according to the different types of the second beam.

For example, if the second beam is visible light, the optical identifier may directly take pictures. The operator or user may identify the marker 6 directly from the picture taken by the optical identifier. Alternatively, if the second beam is visible light, the optical identifier may be an integrated equipment which is directly combined with an image obtaining device and a display device (e.g. LCD display, LED display and OLED display) and which may directly display the marker 6 in the detected subject 7 via the display screen. Certainly, further, the tracing device 100 may also incorporate a computer and programs to process the obtained images, for more precisely and positively confirming the marker 6. In this case, the processing may be rotating, reversing, distorting, cropping and coloring (e.g. in green, for increasing contrast) of the image, etc. For example, if the second beam is invisible light, the optical identifier is required to acquire the invisible light and then convert the image information it represents into an image within the range of visible light, for the operator to view. In addition, for the purpose of observation, the image of the marker 6 may also be fused with the detected subject 7 so as to observe the marker 6 together with the detected subject 7. For example, the image information represented by the second beam is extracted, fused and colored (i.e. in a color manifested by the fluorescence after coloring, which may be configured in any color) by software. The color manifested by the fluorescence after coloring is configured by the operator according to his/her habit and then output by a medically dedicated display. A conventional configuration may be coloring in green which increases the contrast and may help identifying the testing boundary.

During discontinuous recording process, the optical identifier may be used to take pictures. If a continuous recording process is desired to obtain, the optical identifier may be used to take dynamic graphics or continuous images. For example, in order to observe the motion pattern and status, etc. of the marker 6 expected to be observed within a period of time, the optical identifier is configured to be a video camera/vidicon.

In an example, the optical identifier includes a camera (which can take pictures or images). The camera has a photosensitive element/photosensitive sensor, e.g. CMOS, CCD or other suitable types of image sensors.

As required, one or more cameras may be included. The number of cameras may be related to the volume and size of the detected subject 7 and the marker 6, and may also be related to the position where the optical identifier is placed. For example, if the detected subject 7 is relatively large, and the size of the optical identifier is relatively small and cannot well cover the area to be detected (and therefore may not be able to show the complete image of the marker 6), providing a plurality of cameras is easy to implement and required to be taken into special consideration.

As aforementioned, the types of the second beam may be associated with the types of the camera, but the camera is at least sensitive to the second beam, that is, the camera can acquire the second beam and generate an image according to the second beam. Optionally, the tracing device 100 includes a plurality of cameras (e.g. two, three, four or even more cameras), and the plurality of cameras have at least a first camera and a second camera, wherein the first camera is a camera for visible light and the second camera is a camera for near infrared light. The two cameras may operate or not operate independently from each other. Specifically, they may be configured as needed.

In some other examples, the tracing device 100 includes at least two cameras. The at least two cameras include a first camera operating in the region of near infrared light and a second camera operating in the visible region which can operate optionally independently from each other. Accordingly, the tracing device 100 includes a first working mode and a second working mode, which are optionally executed. In the above, under the first working mode, the light source operates in a state of being constantly bright in which the light source continuously emits the first beam and the first camera and the second camera operate simultaneously. Under the second working mode, the light source operates in a pulse state in which the light source intermittently emits the first beam and at least the first camera operates.

In some other alternative examples adjusted as required, the optical identifier includes an optical component configured to increase permeability of and/or filter the second beam before the second beam reaches the cameras. That is, the optical identifier includes an optical component. The optical component may be equipped with different functionalized members and electronic parts and components according to different functional requirements.

For example, based on the requirement of reducing light loss (which may also be partially avoided by increasing the space of the optical identifier for receiving the second beam), the optical component includes an anti-reflection means. Exemplarily, the anti-reflection means includes an anti-reflection film or anti-reflection lens 3 (which may increase permeability of light waves at 400-900 nm). For the issue that the light wave of the second beam may be impure, the optical component includes a light filter 2. For example, it is selected as a band-pass light filter 2, a light filter 2 which only allows light at 785±6 nm to pass. Simultaneously, the light filter 2 keeps light at 785±10 nm from passing the light filter 2. Alternatively, the optical component may have both an anti-reflection means and a light filter 2. And the light filter 2 is located between the cameras and the anti-reflection means. The anti-reflection means includes an anti-reflection film or anti-reflection lens 3. In this way, the second beam may be subjected to filtering which makes it monochromatic light having a higher purity before increasing permeability and then entering into the photosensitive element of the optical identifier for optical collection. In an example, the cameras have a preset wavelength collection range. And the wavelength collection range is from 400 to 900 nm so as to further receive (not lose) image information in the second beam.

Based on the aforementioned tracing device, a tracing method is also provided in the examples. In other words, the tracing method can be implemented by at least one of the aforementioned optional tracing devices.

Referring to FIG. 2, the tracing method includes:

using the first beam emitted from the light source to irradiate the detected subject, in which case the tracer in the detected subject can generate a second beam under the excitation of the first beam; wherein normally a tracer (contrast medium) is introduced into the detected subject invasively or non-invasively in a suitable way in advance, the detected subject may be e.g. an in vitro tissue or an abiotic animal body; and

receiving, by the optical identifier, the second beam generated by the tracer in the detected subject so as to show an image of the marker different from the detected subject.

The marker in the detected subject is only a part of the detected subject. Therefore, actually, the detected subject has a marker area (such as a lesion area, e.g. tumor area) and a non-marker area (non-lesion area, normal tissue area). The marker may absorb and keep the tracer, while other non-markers cannot absorb or keep the tracer. Therefore, when the first beam irradiates the lesion area, the lesion area luminesces (generates the second beam which is visible or invisible to naked eyes), while the non-lesion areas near or around it do not luminesce (at least do not generate the second beam). Thereby, difference in brightness can be found in the image (for example, a gray scale image/gray level image with gray level difference). In the above, the marker (e.g. tumor) may be represented by the bright part or by the dark part, which may be adjusted as required by the design. That is, the bright part and the dark part in the gray level image are distinguished and divided by the fact whether they emit the second beam or not.

For a detected subject in which the existence of a marker is unknown, the method proposed by the present disclosure is used to run a test which is to qualitatively learn whether the detected subject has a marker by taking pictures. If image information e.g. outline and shape denoting the marker, can be acquired from the taken picture, one may determine that there is a marker. If one fails to identify image information e.g. outline and shape denoting the marker, the possible reason may be overtime detection, or too small or too large tracer dose, or too short or too long administration time, etc. Therefore, in the case where one does not identify image information e.g. outline and shape denoting the marker from the taken picture, in order to confirm that the detected subject does not have a marker, one may need to do a large number of targeted experiments on different detected subjects for verification or confirmation, so as to ensure that the tracer is in a sufficient and effective amount throughout the detection. In the above, “sufficient and effective amount” means that when the detected subject has a marker, the administered tracer may be absorbed and kept by the marker in the detection process.

Alternatively, for a detected subject which is known to have a marker, the method proposed by the present disclosure is used to run a test which is to determine the relative position of the marker in the detected subject by taking pictures. In other words, for a detected subject which has been confirmed to have a marker (e.g. by blood examination indicator or clinical symptoms or physiological condition), the method of the present disclosure may be used to obtain the position of the lesion. That is, the method of the present disclosure may be used to indicate the position information of the lesion. It is not expected to be able to provide specific diagnosis information only with such position information, but it generally may be used as an intermediate result.

In addition, in some examples, for a detected subject in which the existence of e.g. tumor (marker) is known or unknown, even the detected subject is confirmed to have tumor cells, tissues or lesions by the method of the present disclosure, it does not necessarily mean that the detected subject has cancer.

The present disclosure provides the following exemplary beneficial effects.

The tracing device and method provided by the embodiments of the present disclosure generate, by the interaction of a light ray with the detected subject, a detectable and new light ray, and then realize identification of markers with the detectable and new light ray. With a relatively simple structure, the device finds an easier and simpler implementation in equipment such as CT and MRI.

Although the present disclosure has been explained and described with specific examples, it should be appreciated that many other changes and modifications may be made without departing from the spirit and scope of the present disclosure. Therefore, it means that all such changes and modifications falling within the scope of the present disclosure are covered by the appended claims.

### INDUSTRIAL APPLICABILITY

The tracing device and tracing method provided by the present disclosure can identify markers in the detected subject by the photoluminescence of the tracer. The tracing device of the present disclosure is highly sensitive and has simple structure. The tracing method of the present disclosure is easy to implement and can identify markers clearly.

1. A tracing device, configured to showing a marker in a detected subject, wherein the tracing device comprises:
a light source, the light source being configured to provide a first beam to irradiate the detected subject, wherein the first beam can interact with the detected subject and then the detected subject generates a second beam different from the first beam; and
an optical identifier, the optical identifier being configured to detect the second beam so as to identify the marker on the detected subject.

2. The tracing device according to claim 1, wherein the first beam and the second beam are not consistent in wavelength; and
the light source can be configured such that the first beam obliquely irradiates the detected subject, and the optical identifier can be configured to directly face the detected subject so as to obtain an image of the marker in a front-view direction.

3. The tracing device according to claim 1, wherein the first beam is laser and the first beam has a wavelength of 779 to 791 nm.

4. The tracing device according to claim 1, wherein the second beam is fluorescence and the second beam has a wave peak with a central wavelength of 810 to 830 nm.

5. The tracing device according to claim 1, wherein the light source comprises a light provider and a filter compatible with each other, the filter is configured to filter light generated by the light provider in terms of wavelength, so as to generate the first beam.

6. The tracing device according to claim 5, wherein the light source further comprises an emergent light head, the emergent light head is cylindrical and sleeved on the light provider.

7. The tracing device according to claim 5, wherein the tracing device further comprises an adjuster, the adjuster is configured to be able to move the light provider so as to change a position of the light provider relative to the detected area.

8. The tracing device according to claim 7, wherein the adjuster is a mechanical arm, and the light provider is provided on the mechanical arm;
or,
the adjuster is a convex lens or a concave lens for the first beam to pass, and the position of the adjuster relative to the light provider is adjustable.

9. The tracing device according to claim 1, wherein the light source has an intensity less than 0.499 W.

10. The tracing device according to claim 1, wherein the optical identifier comprises a plurality of cameras, and the plurality of cameras comprise at least a first camera and a second camera, wherein the first camera is a camera for visible light and the second camera is a camera for near infrared light.

11. The tracing device according to claim 10, wherein the at least two cameras comprise a first camera operating in a near infrared region, and a second camera operating in a visible region, which can operate independently from each other, and the tracing device comprises a first working mode and a second working mode to be executed;
under the first working mode, the light source operates in a state of being constantly bright in which the light source continuously emits the first beam, and the first camera and the second camera operate simultaneously; and
under the second working mode, the light source operates in a pulse state in which the light source intermittently emits the first beam, and at least the first camera operates.

12. The tracing device according to claim 10, wherein the optical identifier comprises an optical component which is configured to perform permeability improvement and/or filtering processing to the second beam before the second beam reaches the cameras.

13. The tracing device according to claim 12, wherein the optical component comprises an anti-reflection means, the anti-reflection means comprises an anti-reflection film or anti-reflection lens;
or
the optical component comprises a light filter;
or
the optical component comprises an anti-reflection means and a light filter, and the light filter is located between the cameras and the anti-reflection means, and the anti-reflection means comprises an anti-reflection film or anti-reflection lens.

14. The tracing device according to claim 10, wherein the cameras have a wavelength collection range of 400 to 900 nm.

15. The tracing device according to claim 1, wherein the tracing device further comprises a controller, and the controller is electrically connected with the light source so as to control an emission frequency of the light source.

16. The tracing device according to claim 1, wherein the first beam is shaped as point light, ceiling light, lattice light or linear light.

17. The tracing device according to claim 5, wherein the light source further comprises a power supply electrically connected with the light provider, and the power supply is a battery or a power adapter.

18. A tracing method, the tracing method being able to be implemented by the tracing device according to claim 1, wherein the tracing method comprises:
using the first beam emitted from the light source to irradiate the detected subject, in which case a tracer in the detected subject can generate the second beam under excitation of the first beam; and
receiving, by the optical identifier, the second beam generated by the tracer in the detected subject so as to show an image of the marker different from the detected subject.

19. The tracing method according to claim 18, wherein the tracer is indocyanine green.

20. The tracing method according to claim 18, wherein in using the first beam emitted from the light source to irradiate the detected subject, the first beam obliquely irradiates an area to be detected of the detected subject; and the optical identifier directly faces the area to be detected when receiving the second beam.
